(19) United States
(12) Patent Application Publication
(10) Pub. No.: US 2023/0240374 A1
(43) Pub. Date: Aug. 3, 2023

(54) ELECTRONIC VAPOR PROVISION SYSTEM AND METHOD

(71) Applicant: NICOVENTURES TRADING LIMITED, London (GB)

(72) Inventor: Charles Jacob Novak, III, Winston-Salem, NC (US)

(21) Appl. No.: 17/649,205

(22) Filed: Jan. 28, 2022

(51) Int. Cl. A24F 40/51; A24F 40/60; A24F 40/40; A24F 40/57; A24F 40/65; A61B 5/16; A61B 5/22

(52) U.S. Cl. CPC A24F 40/51; A24F 40/60; A24F 40/40; A24F 40/57; A24F 40/65; A61B 5/165; A61B 5/225

(57) ABSTRACT

An electronic vapor provision system (EVPS) can include an electronic vapor provision device; at least a first sensor configured to generate information about how firmly a user is holding the device; and a control processor configured to modify one or more operational parameters of the EVPS responsive to the information about how firmly the user is holding the device.

### TECHNICAL FIELD

The present disclosure relates to an electronic vapor provision system and method.

### BACKGROUND

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly or impliedly admitted as prior art against the present disclosure.

Aerosol provision systems (or equally, electronic vapor provision systems) are popular with users as they enable the delivery of active ingredients (such as nicotine) to the user in a convenient manner and on demand.

As an example of an aerosol provision system, electronic cigarettes (e-cigarettes) generally contain a reservoir of a source liquid containing a formulation, typically including nicotine, from which an aerosol is generated, e.g. through heat vaporization. An aerosol source for an aerosol provision system may thus comprise a heater having a heating element arranged to receive source liquid from the reservoir, for example through wicking/capillary action. Other source materials may be similarly heated to create an aerosol, such as botanical matter, or a gel comprising an active ingredient and/or flavoring. Hence more generally, the e-cigarette may be thought of as comprising or receiving a payload for heat vaporization.

While a user inhales on the device, electrical power is supplied to the heating element to vaporize the aerosol source (a portion of the payload) in the vicinity of the heating element, to generate an aerosol for inhalation by the user. Such devices are usually provided with one or more air inlet holes located away from a mouthpiece end of the system. When a user sucks on a mouthpiece connected to the mouthpiece end of the system, air is drawn in through the inlet holes and past the aerosol source. There is a flow path connecting between the aerosol source and an opening in the mouthpiece so that air drawn past the aerosol source continues along the flow path to the mouthpiece opening, carrying some of the aerosol from the aerosol source with it. The aerosol-carrying air exits the aerosol provision system through the mouthpiece opening for inhalation by the user.

Usually an electric current is supplied to the heater when a user is drawing/puffing on the device. Typically, the electric current is supplied to the heater, e.g. resistance heating element, in response to either the activation of an airflow sensor along the flow path as the user inhales/draw/puffs or in response to the activation of a button by the user. The heat generated by the heating element is used to vaporize a formulation. The released vapor mixes with air drawn through the device by the puffing consumer and forms an aerosol. Alternatively or in addition, the heating element is used to heat but typically not burn a botanical such as tobacco, to release active ingredients thereof as a vapor/aerosol.

It will be appreciated that as a frequently used electronic device that, in use, draws sufficient current from its battery to heat a portion of payload to the point where it generates a vapor, it is beneficial if the device activates in a manner that is convenient for the user and its use, and also preferably does so in a manner that is beneficial to the battery and/or other workings of the device.

### SUMMARY

The present disclosure seeks to address or mitigate the aforementioned need.

Various aspects and features of the present disclosure are defined in the appended claims and within the text of the accompanying description.

In a first aspect, an electronic vapor provision system ‘EVPS’ is provided.

In another aspect, a method of electronic vapor provision is provided.

It is to be understood that both the foregoing general description of the disclosure and the following detailed description are exemplary, but are not restrictive, of the invention.

### BRIEF DESCRIPTION OF THE DRAWINGS

A more complete appreciation of the disclosure and many of the attendant advantages thereof will be readily obtained as the same becomes better understood by reference to the following detailed description when considered in connection with the accompanying drawings, wherein:

FIG. 1 is a schematic diagram of a vapor/aerosol provision system, in accordance with embodiments of the present disclosure.

FIG. 2 is a schematic diagram of the body 20 of the system of FIG. 1, in accordance with embodiments of the present disclosure.

FIG. 3 is a schematic diagram of a cartomizer 30 of the system of FIG. 1, in accordance with embodiments of the disclosure.

FIG. 4 is a schematic diagram a connector of the system of FIG. 1, in accordance with embodiments of the disclosure.

FIG. 5A is a schematic diagram of functional components of the system of FIG. 1, in accordance with embodiments of the disclosure.

FIG. 5B is a schematic diagram of functional components of a processor of the system of FIG. 1, in accordance with embodiments of the disclosure.

FIG. 6 is a schematic diagram of a delivery ecosystem, in accordance with embodiments of the disclosure.

FIG. 7 is a schematic diagram of functional components of a mobile communication device, in accordance with embodiments of the disclosure.

FIG. 8 is a flow diagram of a method of electronic vapor provision, in accordance with embodiments of the disclosure.

### DESCRIPTION OF EMBODIMENTS

An electronic vapor provision system and method are disclosed. In the following description, a number of specific details are presented in order to provide a thorough understanding of the embodiments of the present disclosure. It will be apparent, however, to a person skilled in the art that these specific details need not be employed to practice the present invention. Conversely, specific details known to the person skilled in the art are omitted for the purposes of clarity where appropriate. Aerosol provision systems (or equally, electronic vapor provision systems) are similar terms for a delivery device for a user.

The term ‘delivery device’ and by extension ‘aerosol provision system’ or ‘electronic vapor provision system’ may encompass systems that deliver a least one substance to a user, and include non-combustible aerosol provision systems that release compounds from an aerosol-generating material without combusting the aerosol-generating material, such as electronic cigarettes, tobacco heating products, and hybrid systems to generate aerosol using a combination of aerosol-generating materials, wherein the at least one substance may or may not comprise nicotine.

The substance to be delivered may be an aerosol-generating material and as appropriate, may comprise one or more active constituents, one or more flavors, one or more aerosol-former materials, and/or one or more other functional materials.

Currently, the most common example of such a delivery device is an aerosol provision system (e.g. a non-combustible aerosol provision system) or electronic vapor provision system (EVPS), such as an e-cigarette. Throughout the following description the term “e-cigarette” is sometimes used but this term may be used interchangeably with these terms above except where stated otherwise or where context indicates otherwise. Similarly the terms ‘vapor’ and ‘aerosol’ are referred to equivalently herein.

Generally, the electronic vapor/aerosol provision system may be an electronic cigarette, also known as a vaping device or electronic nicotine delivery device (END), although it is noted that the presence of nicotine in the aerosol-generating (e.g. aerosolizable) material is not a requirement. In some embodiments, a non-combustible aerosol provision system is a tobacco heating system, also known as a heat-not-burn system. An example of such a system is a tobacco heating system. In some embodiments, the non-combustible aerosol provision system is a hybrid system to generate aerosol using a combination of aerosol-generating materials, one or a plurality of which may be heated. Each of the aerosol-generating materials may be, for example, in the form of a solid, liquid or gel and may or may not contain nicotine. In some embodiments, the hybrid system comprises a liquid or gel aerosol-generating material and a solid aerosol-generating material. The solid aerosol-generating material may comprise, for example, tobacco or a non-tobacco product. Meanwhile in some embodiments, the non-combustible aerosol provision system generates a vapor/aerosol from one or more such aerosol-generating materials.

Typically, the non-combustible aerosol provision system may comprise a non-combustible aerosol provision device and an article (otherwise referred to as a consumable) for use with the non-combustible aerosol provision system. However, it is envisaged that articles which themselves comprise a means for powering an aerosol generating component (e.g. an aerosol generator such as a heater, vibrating mesh or the like) may themselves form the non-combustible aerosol provision system. In one embodiment, the non-combustible aerosol provision device may comprise a power source and a controller. The power source may be an electric power source or an exothermic power source. In one embodiment, the exothermic power source comprises a carbon substrate which may be energized so as to distribute power in the form of heat to an aerosolizable material or heat transfer material in proximity to the exothermic power source. In one embodiment, the power source, such as an exothermic power source, is provided in the article so as to form the non-combustible aerosol provision. In one embodiment, the article for use with the non-combustible aerosol provision device may comprise an aerosolizable material.

In some embodiments, the aerosol generating component is a heater capable of interacting with the aerosolizable material so as to release one or more volatiles from the aerosolizable material to form an aerosol. In one embodiment, the aerosol generating component is capable of generating an aerosol from the aerosolizable material without heating. For example, the aerosol generating component may be capable of generating an aerosol from the aerosolizable material without applying heat thereto, for example via one or more of vibrational, mechanical, pressurization or electrostatic means.

In some embodiments, the aerosolizable material may comprise an active material, an aerosol forming material and optionally one or more functional materials. The active material may comprise nicotine (optionally contained in tobacco or a tobacco derivative) or one or more other non-olfactory physiologically active materials. A non-olfactory physiologically active material is a material which is included in the aerosolizable material in order to achieve a physiological response other than olfactory perception. The aerosol forming material may comprise one or more of glycerine, glycerol, propylene glycol, diethylene glycol, triethylene glycol, tetraethylene glycol, 1,3-butylene glycol, erythritol, meso-Erythritol, ethyl vanillate, ethyl laurate, a diethyl suberate, triethyl citrate, triacetin, a diacetin mixture, benzyl benzoate, benzyl phenyl acetate, tributyrin, lauryl acetate, lauric acid, myristic acid, and propylene carbonate. The one or more functional materials may comprise one or more of flavors, carriers, pH regulators, stabilizers, and/or antioxidants.

In some embodiments, the article for use with the non-combustible aerosol provision device may comprise aerosolizable material or an area for receiving aerosolizable material. In one embodiment, the article for use with the non-combustible aerosol provision device may comprise a mouthpiece. The area for receiving aerosolizable material may be a storage area for storing aerosolizable material. For example, the storage area may be a reservoir. In one embodiment, the area for receiving aerosolizable material may be separate from, or combined with, an aerosol generating area.

The aerosol provision system need not provide the aerosol directly to the user, but may provide it to an intermediary device or conveyor that causes/enables the introduction of an active ingredient into the body of the user in a manner that allows the active ingredient to take effect.

An example may thus include a device that disperses an aerosol into a receptacle, after which a user may take the receptacle from the device and inhale or sip the aerosol. Hence the delivery device does not necessarily have to be directly engaged with by the user at the point of consumption.

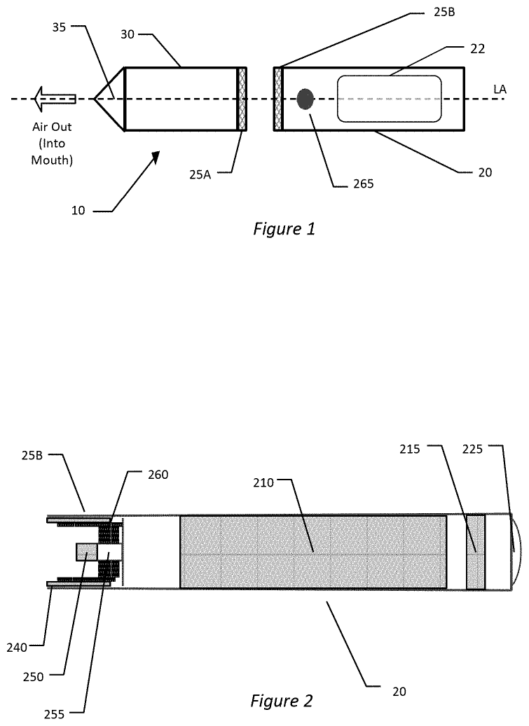

Referring now to the drawings, wherein like reference numerals designate identical or corresponding parts throughout the several views, FIG. 1 is a schematic diagram of a vapor/aerosol provision system such as an e-cigarette 10 (not to scale), providing a non-limiting example of a delivery device in accordance with some embodiments of the disclosure.

The e-cigarette has a generally cylindrical shape, extending along a longitudinal axis indicated by dashed line LA, and comprises two main components, namely a body 20 and a cartomizer 30. The cartomizer includes an internal chamber containing a reservoir of a payload such as for example a liquid comprising nicotine, a vaporizer (such as a heater), and a mouthpiece 35. References to ‘nicotine’ hereafter will be understood to be merely an example and can be substituted with any suitable active ingredient. References to ‘liquid’ as a payload hereafter will be understood to be merely an example and can be substituted with any suitable payload such as botanical matter (for example tobacco that is to be heated rather than burned), or a gel comprising an active ingredient and/or flavoring. The reservoir may be a foam matrix or any other structure for retaining the liquid until such time that it is required to be delivered to the vaporizer. In the case of a liquid/flowing payload, the vaporizer is for vaporizing the liquid, and the cartomizer 30 may further include a wick or similar facility to transport a small amount of liquid from the reservoir to a vaporizing location on or adjacent the vaporizer. In the following, a heater is used as a specific example of a vaporizer. However, it will be appreciated that other forms of vaporizer (for example, those which utilize ultrasonic waves) could also be used and it will also be appreciated that the type of vaporizer used may also depend on the type of payload to be vaporized.

The body 20 includes a re-chargeable cell or battery to provide power to the e-cigarette 10 and a circuit board for generally controlling the e-cigarette. When the heater receives power from the battery, as controlled by the circuit board, the heater vaporizes the liquid and this vapor is then inhaled by a user through the mouthpiece 35. In some specific embodiments the body is further provided with a manual activation device 265, e.g. a button, switch, or touch sensor located on the outside of the body.

The body 20 and cartomizer 30 may be detachable from one another by separating in a direction parallel to the longitudinal axis LA, as shown in FIG. 1, but are joined together when the device 10 is in use by a connection, indicated schematically in FIG. 1 as 25A and 25B, to provide mechanical and electrical connectivity between the body 20 and the cartomizer 30. The electrical connector 25B on the body 20 that is used to connect to the cartomizer 30 also serves as a socket for connecting a charging device (not shown) when the body 20 is detached from the cartomizer 30. The other end of the charging device may be plugged into a USB socket to re-charge the cell in the body 20 of the e-cigarette 10. In other implementations, a cable may be provided for direct connection between the electrical connector 25B on the body 20 and a USB socket.

The e-cigarette 10 is provided with one or more holes (not shown in FIG. 1) for air inlets. These holes connect to an air passage through the e-cigarette 10 to the mouthpiece 35. When a user inhales through the mouthpiece 35, air is drawn into this air passage through the one or more air inlet holes, which are suitably located on the outside of the e-cigarette. When the heater is activated to vaporize the nicotine from the cartridge, the airflow passes through, and combines with, the generated vapor, and this combination of airflow and generated vapor then passes out of the mouthpiece 35 to be inhaled by a user. Except in single-use devices, the cartomizer 30 may be detached from the body 20 and disposed of when the supply of liquid is exhausted (and replaced with another cartomizer if so desired).

It will be appreciated that the e-cigarette 10 shown in FIG. 1 is presented by way of example, and various other implementations can be adopted. For example, in some embodiments, the cartomizer 30 is provided as two separable components, namely a cartridge comprising the liquid reservoir and mouthpiece (which can be replaced when the liquid from the reservoir is exhausted), and a vaporizer comprising a heater (which is generally retained). As another example, the charging facility may connect to an additional or alternative power source, such as a car cigarette lighter.

FIG. 2 is a schematic (simplified) diagram of the body 20 of the e-cigarette 10 of FIG. 1 in accordance with some embodiments of the disclosure. FIG. 2 can generally be regarded as a cross-section in a plane through the longitudinal axis LA of the e-cigarette 10. Note that various components and details of the body, e.g. such as wiring and more complex shaping, have been omitted from FIG. 2 for reasons of clarity.

The body 20 includes a battery or cell 210 for powering the e-cigarette 10 in response to a user activation of the device. Additionally, the body 20 includes a control unit (not shown in FIG. 2), for example a chip such as an application specific integrated circuit (ASIC) or microcontroller, for controlling the e-cigarette 10. The microcontroller or ASIC includes a CPU or micro-processor. The operations of the CPU and other electronic components are generally controlled at least in part by software programs running on the CPU (or other component). Such software programs may be stored in non-volatile memory, such as ROM, which can be integrated into the microcontroller itself, or provided as a separate component. The CPU may access the ROM to load and execute individual software programs as and when required. The microcontroller also contains appropriate communications interfaces (and control software) for communicating as appropriate with other devices in the body 10.

The body 20 further includes a cap 225 to seal and protect the far (distal) end of the e-cigarette 10. Typically there is an air inlet hole provided in or adjacent to the cap 225 to allow air to enter the body 20 when a user inhales on the mouthpiece 35. The control unit or ASIC may be positioned alongside or at one end of the battery 210. In some embodiments, the ASIC is attached to a sensor unit 215 to detect an inhalation on mouthpiece 35 (or alternatively the sensor unit 215 may be provided on the ASIC itself). In either case, the sensor unit 215, with or without the ASIC, may be understood as an example of a sensor platform. An air path is provided from the air inlet through the e-cigarette, past the airflow sensor 215 and the heater (in the vaporizer or cartomizer 30), to the mouthpiece 35. Thus when a user inhales on the mouthpiece of the e-cigarette, the CPU detects such inhalation based on information from the airflow sensor 215.

At the opposite end of the body 20 from the cap 225 is the connector 25B for joining the body 20 to the cartomizer 30. The connector 25B provides mechanical and electrical connectivity between the body 20 and the cartomizer 30. The connector 25B includes a body connector 240, which is metallic (silver-plated in some embodiments) to serve as one terminal for electrical connection (positive or negative) to the cartomizer 30. The connector 25B further includes an electrical contact 250 to provide a second terminal for electrical connection to the cartomizer 30 of opposite polarity to the first terminal, namely body connector 240. The electrical contact 250 is mounted on a coil spring 255. When the body 20 is attached to the cartomizer 30, the connector 25A on the cartomizer 30 pushes against the electrical contact 250 in such a manner as to compress the coil spring in an axial direction, i.e. in a direction parallel to (co-aligned with) the longitudinal axis LA. In view of the resilient nature of the spring 255, this compression biases the spring 255 to expand, which has the effect of pushing the electrical contact 250 firmly against connector 25A of the cartomizer 30, thereby helping to ensure good electrical connectivity between the body 20 and the cartomizer 30. The body connector 240 and the electrical contact 250 are separated by a trestle 260, which is made of a non-conductor (such as plastic) to provide good insulation between the two electrical terminals. The trestle 260 is shaped to assist with the mutual mechanical engagement of connectors 25A and 25B.

As mentioned above, a button 265, which represents a form of manual activation device 265, may be located on the outer housing of the body 20. The button 265 may be implemented using any appropriate mechanism which is operable to be manually activated by the user—for example, as a mechanical button or switch, a capacitive or resistive touch sensor, and so on. It will also be appreciated that the manual activation device 265 may be located on the outer housing of the cartomizer 30, rather than the outer housing of the body 20, in which case, the manual activation device 265 may be attached to the ASIC via the connections 25A, 25B. The button 265 might also be located at the end of the body 20, in place of (or in addition to) cap 225.

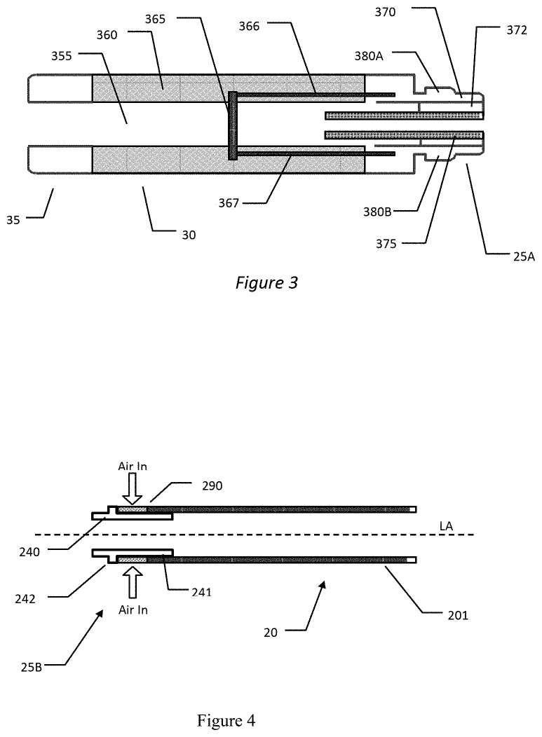

FIG. 3 is a schematic diagram of the cartomizer 30 of the e-cigarette 10 of FIG. 1 in accordance with some embodiments of the disclosure. FIG. 3 can generally be regarded as a cross-section in a plane through the longitudinal axis LA of the e-cigarette 10. Note that various components and details of the cartomizer 30, such as wiring and more complex shaping, have been omitted from FIG. 3 for reasons of clarity.

The cartomizer 30 includes an air passage 355 extending along the central (longitudinal) axis of the cartomizer 30 from the mouthpiece 35 to the connector 25A for joining the cartomizer 30 to the body 20. A reservoir of liquid 360 is provided around the air passage 335. This reservoir 360 may be implemented, for example, by providing cotton or foam soaked in liquid. The cartomizer 30 also includes a heater 365 for heating liquid from reservoir 360 to generate vapor to flow through air passage 355 and out through mouthpiece 35 in response to a user inhaling on the e-cigarette 10. The heater 365 is powered through lines 366 and 367, which are in turn connected to opposing polarities (positive and negative, or vice versa) of the battery 210 of the main body 20 via connector 25A (the details of the wiring between the power lines 366 and 367 and connector 25A are omitted from FIG. 3).

The connector 25A includes an inner electrode 375, which may be silver-plated or made of some other suitable metal or conducting material. When the cartomizer 30 is connected to the body 20, the inner electrode 375 contacts the electrical contact 250 of the body 20 to provide a first electrical path between the cartomizer 30 and the body 20. In particular, as the connectors 25A and 25B are engaged, the inner electrode 375 pushes against the electrical contact 250 so as to compress the coil spring 255, thereby helping to ensure good electrical contact between the inner electrode 375 and the electrical contact 250.

The inner electrode 375 is surrounded by an insulating ring 372, which may be made of plastic, rubber, silicone, or any other suitable material. The insulating ring is surrounded by the cartomizer connector 370, which may be silver-plated or made of some other suitable metal or conducting material. When the cartomizer 30 is connected to the body 20, the cartomizer connector 370 contacts the body connector 240 of the body 20 to provide a second electrical path between the cartomizer 30 and the body 20. In other words, the inner electrode 375 and the cartomizer connector 370 serve as positive and negative terminals (or vice versa) for supplying power from the battery 210 in the body 20 to the heater 365 in the cartomizer 30 via supply lines 366 and 367 as appropriate.

The cartomizer connector 370 is provided with two lugs or tabs 380A, 380B, which extend in opposite directions away from the longitudinal axis of the e-cigarette 10. These tabs are used to provide a bayonet fitting in conjunction with the body connector 240 for connecting the cartomizer 30 to the body 20. This bayonet fitting provides a secure and robust connection between the cartomizer 30 and the body 20, so that the cartomizer and body are held in a fixed position relative to one another, with minimal wobble or flexing, and the likelihood of any accidental disconnection is very small. At the same time, the bayonet fitting provides simple and rapid connection and disconnection by an insertion followed by a rotation for connection, and a rotation (in the reverse direction) followed by withdrawal for disconnection. It will be appreciated that other embodiments may use a different form of connection between the body 20 and the cartomizer 30, such as a snap fit or a screw connection.

FIG. 4 is a schematic diagram of certain details of the connector 25B at the end of the body 20 in accordance with some embodiments of the disclosure (but omitting for clarity most of the internal structure of the connector as shown in FIG. 2, such as trestle 260). In particular, FIG. 4 shows the external housing 201 of the body 20, which generally has the form of a cylindrical tube. This external housing 201 may comprise, for example, an inner tube of metal with an outer covering of paper or similar. The external housing 201 may also comprise the manual activation device 265 (not shown in FIG. 4) so that the manual activation device 265 is easily accessible to the user.

The body connector 240 extends from this external housing 201 of the body 20. The body connector 240 as shown in FIG. 4 comprises two main portions, a shaft portion 241 in the shape of a hollow cylindrical tube, which is sized to fit just inside the external housing 201 of the body 20, and a lip portion 242 which is directed in a radially outward direction, away from the main longitudinal axis (LA) of the e-cigarette. Surrounding the shaft portion 241 of the body connector 240, where the shaft portion does not overlap with the external housing 201, is a collar or sleeve 290, which is again in a shape of a cylindrical tube. The collar 290 is retained between the lip portion 242 of the body connector 240 and the external housing 201 of the body, which together prevent movement of the collar 290 in an axial direction (i.e. parallel to axis LA). However, collar 290 is free to rotate around the shaft portion 241 (and hence also axis LA).

As mentioned above, the cap 225 is provided with an air inlet hole to allow air to flow when a user inhales on the mouthpiece 35. However, in some embodiments the majority of air that enters the device when a user inhales flows through collar 290 and body connector 240 as indicated by the two arrows in FIG. 4.

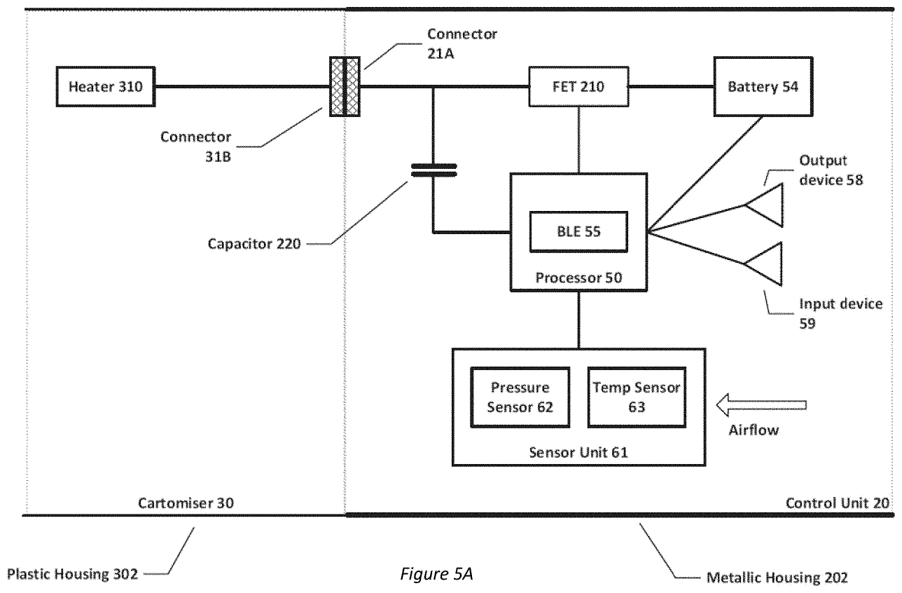

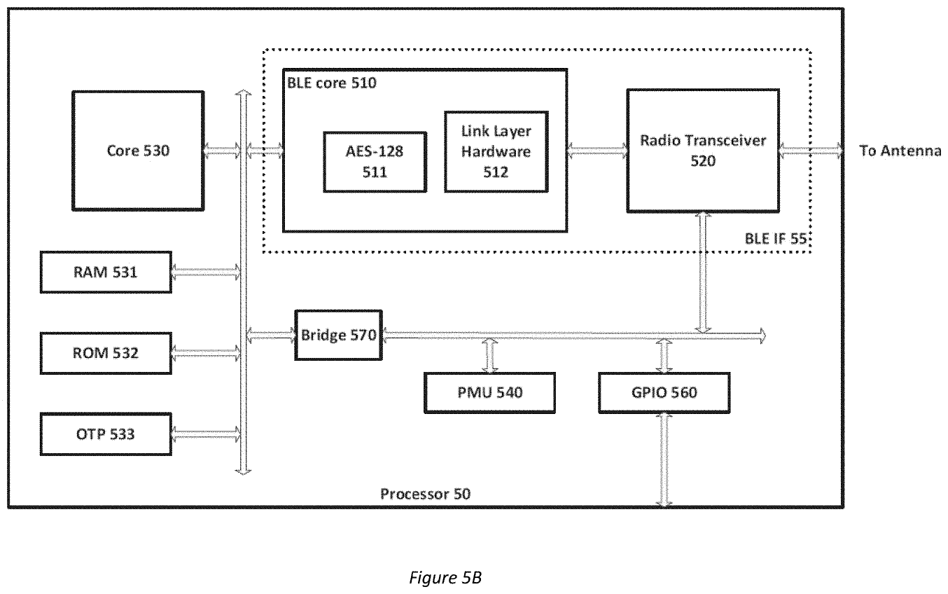

FIG. 5A is a schematic diagram of the main functional components of the e-cigarette 10 of FIG. 1 in accordance with some embodiments of the disclosure. Notably FIG. 5A is primarily concerned with electrical connectivity and functionality—it is not intended to indicate the physical sizing of the different components, nor details of their physical placement within the control unit 20 or cartomizer 30. In addition, it will be appreciated that at least some of the components shown in FIG. 5A located within the control unit 20 may be mounted on the circuit board 28. Alternatively, one or more of such components may instead be accommodated in the control unit to operate in conjunction with the circuit board 28, but not physically mounted on the circuit board itself. For example, these components may be located on one or more additional circuit boards, or they may be separately located (such as battery 54).

As shown in FIG. 5A, the cartomizer contains heater 310 which receives power through connector 31B. The control unit 20 includes an electrical socket or connector 21A for connecting to the corresponding connector 31B of the cartomizer 30 (or potentially to a USB charging device). This then provides electrical connectivity between the control unit 20 and the cartomizer 30.

The control unit 20 further includes a sensor unit 61, which is located in or adjacent to the air path through the control unit 20 from the air inlet(s) to the air outlet (to the cartomizer 30 through the connector 21A). The sensor unit contains a pressure sensor 62 and temperature sensor 63 (also in or adjacent to this air path). The control unit further includes a capacitor 220, a processor 50, a field effect transistor (FET) switch 210, a battery 54, input device 59 (or equivalently 265 in FIG. 1), and output device 58.

The operations of the processor 50 and other electronic components, such as the pressure sensor 62, are generally controlled at least in part by software programs running on the processor (or other components). Such software programs may be stored in non-volatile memory, such as ROM, which can be integrated into the processor 50 itself, or provided as a separate component. The processor 50 may access the ROM to load and execute individual software programs as and when required. The processor 50 also contains appropriate communications facilities, e.g. pins or pads (plus corresponding control software), for communicating as appropriate with other devices in the control unit 20, such as the pressure sensor 62.

The output device(s) 58 may provide visible, audio and/or haptic output. For example, the output device(s) may include a speaker 58, a vibrator, and/or one or more lights. The lights are typically provided in the form of one or more light emitting diodes (LEDs), which may be the same or different colors (or multi-colored). In the case of multi-colored LEDs, different colors are obtained by switching red, green or blue LEDs on, optionally at different relative brightnesses to give corresponding relative variations in color. Where red, green and blue LEDs are provided together, a full range of colors is possible, whilst if only two out of the three red, green and blue LEDs are provided, only a respective sub-range of colors can be obtained.

The output from the output device may be used to signal to the user various conditions or states within the e-cigarette, such as a low battery warning. Different output signals may be used for signaling different states or conditions. For example, if the output device 58 is an audio speaker, different states or conditions may be represented by tones or beeps of different pitch and/or duration, and/or by providing multiple such beeps or tones. Alternatively, if the output device 58 includes one or more lights, different states or conditions may be represented by using different colors, pulses of light or continuous illumination, different pulse durations, and so on. For example, one indicator light might be utilized to show a low battery warning, while another indicator light might be used to indicate that the liquid reservoir 58 is nearly depleted. It will be appreciated that a given e-cigarette may include output devices to support multiple different output modes (audio, visual) etc. The input device(s) 59 may be provided in various forms. For example, an input device (or devices) may be implemented as buttons on the outside of the e-cigarette—e.g. as mechanical, electrical or capacitor (touch) sensors. Some devices may support blowing into the e-cigarette as an input mechanism (such blowing may be detected by pressure sensor 62, which would then be also acting as a form of input device 59), and/or connecting/disconnecting the cartomizer 30 and control unit 20 as another form of input mechanism. Again, it will be appreciated that a given e-cigarette may include input devices 59 to support multiple different input modes.

As noted above, the e-cigarette 10 provides an air path from the air inlet through the e-cigarette, past the pressure sensor 62 and the heater 310 in the cartomizer 30 to the mouthpiece 35. Thus when a user inhales on the mouthpiece of the e-cigarette, the processor 50 detects such inhalation based on information from the pressure sensor 62. In response to such a detection, the CPU supplies power from the battery 54 to the heater, which thereby heats and vaporizes the nicotine from the liquid reservoir 38 for inhalation by the user. Meanwhile for example, for a device which is button activated, a different air path may be used (for example not entering the battery section).

In the particular non-limiting implementation shown in FIG. 5A, a FET 210 is connected between the battery 54 and the connector 21A. This FET 210 acts as a switch. The processor 50 is connected to the gate of the FET to operate the switch, thereby allowing the processor to switch on and off the flow of power from the battery 54 to heater 310 according to the status of the detected airflow. It will be appreciated that the heater current can be relatively large, for example, in the range 1-5 amps, and hence the FET 210 should be implemented to support such current control (likewise for any other form of switch that might be used in place of FET 210).

In order to provide more fine-grained control of the amount of power flowing from the battery 54 to the heater 310, a pulse-width modulation (PWM) scheme may be adopted. A PWM scheme may be based on a repetition period of say 1 ms. Within each such period, the switch 210 is turned on for a proportion of the period, and turned off for the remaining proportion of the period. This is parameterized by a duty cycle, whereby a duty cycle of 0 indicates that the switch is off for all of each period (i.e. in effect, permanently off), a duty cycle of 0.33 indicates that the switch is on for a third of each period, a duty cycle of 0.66 indicates that the switch is on for two-thirds of each period, and a duty cycle of 1 indicates that the FET is on for all of each period (i.e. in effect, permanently on). It will be appreciated that these are only given as example settings for the duty cycle, and intermediate values can be used as appropriate.

The use of PWM provides an effective power to the heater which is given by the nominal available power (based on the battery output voltage and the heater resistance) multiplied by the duty cycle. The processor 50 may, for example, utilize a duty cycle of 1 (i.e. full power) at the start of an inhalation to initially raise the heater 310 to its desired operating temperature as quickly as possible. Once this desired operating temperature has been achieved, the processor 50 may then reduce the duty cycle to some suitable value in order to maintain the heater 310 at the desired operating temperature.

As shown in FIG. 5A, the processor 50 includes a communications interface 55 for wireless communications, in particular, support for Bluetooth® Low Energy (BLE) communications.

Optionally the heater 310 may be utilized as an antenna for use by the communications interface 55 for transmitting and receiving the wireless communications. One motivation for this is that the control unit 20 may have a metal housing 202, whereas the cartomizer portion 30 may have a plastic housing 302 (reflecting the fact that the cartomizer 30 is disposable, whereas the control unit 20 is retained and therefore needs to be more durable). The metal housing acts as a screen or barrier which makes it difficult to locate an antenna within the control unit 20 itself. However, utilizing the heater 310 as the antenna for the wireless communications avoids this metal screening because of the plastic housing of the cartomizer, but without adding additional components or complexity (or cost) to the cartomizer. Alternatively a separate antenna may be provided (not shown), or a portion of the metal housing may be used.

If the heater is used as an antenna then as shown in FIG. 5A, the processor 50, more particularly the communications interface 55, may be coupled to the power line from the battery 54 to the heater 310 (via connector 31B) by a capacitor 220. This capacitive coupling occurs downstream of the switch 210, since the wireless communications may operate when the heater is not powered for heating (as discussed in more detail below). It will be appreciated that capacitor 220 prevents the power supply from the battery 54 to the heater 310 being diverted back to the processor 50.

Note that the capacitive coupling may be implemented using a more complex LC (inductor-capacitor) network, which can also provide impedance matching with the output of the communications interface 55. (As known to the person skilled in the art, this impedance matching supports proper transfer of signals between the communications interface 55 and the heater 310 acting as the antenna, rather than having such signals reflected back along the connection.)

In some implementations, the processor 50 and communications interface are implemented using a Dialog DA14580 chip from Dialog Semiconductor PLC, based in Reading, United Kingdom. Further information (and a data sheet) for this chip is available at: www.dialog-semiconductor.com.

FIG. 5B presents a high-level and simplified overview of this chip 50, including the communications interface 55 for supporting Bluetooth® Low Energy. This interface includes in particular a radio transceiver 520 for performing signal modulation and demodulation, etc., link layer hardware 512, and an advanced encryption facility (128 bits) 511. The output from the radio transceiver 520 is connected to the antenna (for example, to the heater 310 acting as the antenna via capacitive coupling 220 and connectors 21A and 31B).

The remainder of processor 50 includes a general processing core 530, RAM 531, ROM 532, a one-time programming (OTP) unit 533, a general purpose I/O system 560 (for communicating with other components on the PCB 28), a power management unit 540 and a bridge 570 for connecting two buses. Software instructions stored in the ROM 532 and/or OTP unit 533 may be loaded into RAM 531 (and/or into memory provided as part of core 530) for execution by one or more processing units within core 530. These software instructions cause the processor 50 to implement various functionality described herein, such as interfacing with the sensor unit 61 and controlling the heater accordingly. Note that although the device shown in FIG. 5B acts as both a communications interface 55 and also as a general controller for the electronic vapor provision system 10, in other embodiments these two functions may be split between two or more different devices (chips)—e.g. one chip may serve as the communications interface 55, and another chip as the general controller for the electronic vapor provision system 10.

In some implementations, the processor 50 may be configured to prevent wireless communications when the heater is being used for vaporizing liquid from reservoir 38. For example, wireless communications may be suspended, terminated or prevented from starting when switch 210 is switched on. Conversely, if wireless communications are ongoing, then activation of the heater may be prevented—e.g. by discarding a detection of airflow from the sensor unit 61, and/or by not operating switch 210 to turn on power to the heater 310 while the wireless communications are progressing.

One reason for preventing the simultaneous operation of heater 310 for both heating and wireless communications is to avoid any potential interference from the PWM control of the heater. This PWM control has its own frequency (based on the repetition frequency of the pulses), albeit much lower than the frequency of the wireless communications, and the two could potentially interfere with one another. In some situations, such interference may not, in practice, cause any problems, and simultaneous operation of heater 310 for both heating and wireless communications may be allowed (if so desired). This may be facilitated, for example, by techniques such as the appropriate selection of signal strengths and/or PWM frequency, the provision of suitable filtering, etc.

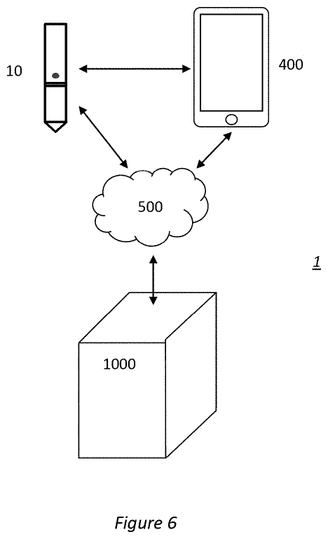

Referring now to FIG. 6, the e-cigarette 10 (or more generally any delivery device as described elsewhere herein) may operate within a wider delivery ecosystem 1.

Within the wider delivery ecosystem, a number of devices may communicate with each other, either directly (for example via BlueTooth®) or indirectly (for example via the internet 500). Examples include but are not limited to a mobile phone 400 and a remote server 1000.

With regards to Bluetooth®, a delivery device 10 may communicate with a mobile communication device using Bluetooth® or Bluetooth® Low Energy communications (or similar schemes) to functionally link the delivery device 10 and an application (app) running on a smartphone 400 or other suitable mobile communication device (tablet, laptop, smartwatch, etc.). Such communications can be used for a wide range of purposes, for example, to upgrade firmware on the e-cigarette 10, to retrieve usage and/or diagnostic data from the e-cigarette 10, to reset or unlock the e-cigarette 10, to control settings on the e-cigarette, etc., or to share processing operations.

In general terms, when the e-cigarette 10 is switched on, such as by using input device 59 (or equivalently 265), or possibly by joining the cartomizer 30 to the control unit 20, it starts to advertise for Bluetooth® Low Energy communication. If this outgoing communication is received by smartphone 400, then the smartphone 400 requests a connection to the e-cigarette 10. The e-cigarette may notify this request to a user via output device 58, and wait for the user to accept or reject the request via input device 59. Assuming the request is accepted, the e-cigarette 10 is able to communicate further with the smartphone 400. Note that the e-cigarette may remember the identity of smartphone 400 and be able to accept future connection requests automatically from that smartphone. Once the connection has been established, the smartphone 400 and the e-cigarette 10 operate in a client-server mode, with the smartphone operating as a client that initiates and sends requests to the e-cigarette which therefore operates as a server (and responds to the requests as appropriate).

A Bluetooth® Low Energy link (also known as Bluetooth Smart®) implements the IEEE 802.15.1 standard, and operates at a frequency of 2.4-2.5 GHz, corresponding to a wavelength of about 12 cm, with data rates of up to 1 Mbit/s. The set-up time for a connection is less than 6 ms, and the average power consumption can be very low—of the order 1 mW or less. A Bluetooth Low Energy link may extend up to some 5 m. However, for the situation shown in FIG. 4, the e-cigarette 10 and the smartphone 400 will typically belong to the same person, and will therefore be in much closer proximity to one another—e.g. 1 m. Further information about Bluetooth Low Energy can be found at: www.bluetooth.com.

It will be appreciated that e-cigarette 10 may support other communications protocols for communication with smartphone 400 (or any other appropriate device). Such other communications protocols may be instead of, or in addition to, Bluetooth Low Energy. Examples of such other communications protocols include Bluetooth® (not the low energy variant), see for example, www.bluetooth.com, near field communications (NFC), as per ISO 13157, and WiFi®. NFC communications operate at much lower wavelengths than Bluetooth (13.56 MHz) and generally have a much shorter range—say <0.2 m. However, this short range is still compatible with many usage scenarios where the user holds or carries both devices. Meanwhile, low-power WiFi® communications, such as IEEE802.11ah, IEEE802.11v, or similar, may be employed between the e-cigarette 10 and a remote device, for example via a wireless access point. In each case, a suitable communications chipset may be included on PCB 28, either as part of the processor 50 or as a separate component. The skilled person will be aware of other wireless communication protocols that may be employed in e-cigarette 10.

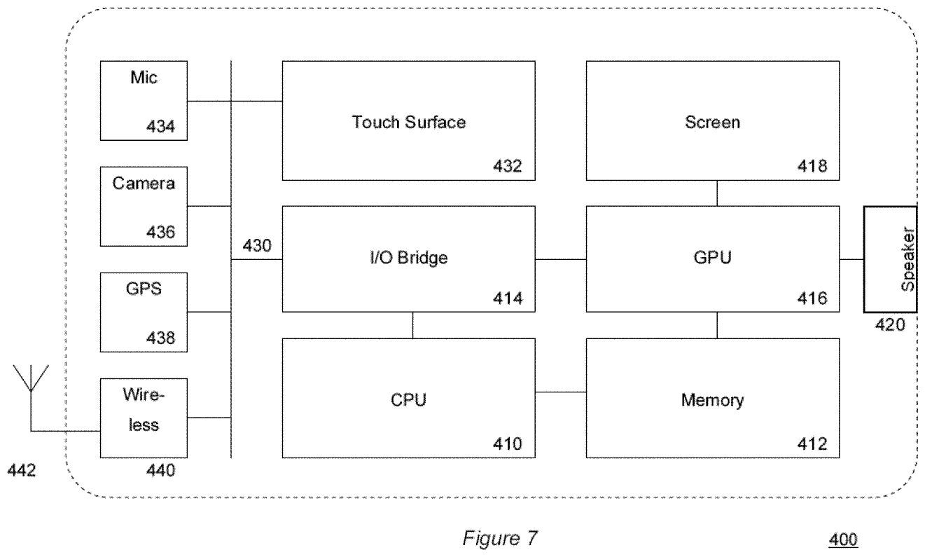

Turning now to FIG. 7, a typical mobile communication device 400 such as a smart phone comprises a central processing unit (CPU) (410). The CPU may communicate with components of the smart phone either through direct connections or via an I/O bridge 414 and/or a bus 430 as applicable.

In the example shown in FIG. 7, the CPU communicates directly with a memory 412, which may comprise a persistent memory such as for example Flash® memory for storing an operating system and applications (apps), and volatile memory such as RAM for holding data currently in use by the CPU. Typically persistent and volatile memories are formed by physically distinct units (not shown). In addition, the memory may separately comprise plug-in memory such as a microSD card, and also subscriber information data on a subscriber information module (SIM) (not shown).

The smart phone may also comprise a graphics processing unit (GPU) 416. The GPU may communicate directly with the CPU or via the I/O bridge, or may be part of the CPU. The GPU may share RAM with the CPU or may have its own dedicated RAM (not shown) and is connected to the display 418 of the mobile phone. The display is typically a liquid crystal (LCD) or organic light-emitting diode (OLED) display, but may be any suitable display technology, such as e-ink. Optionally the GPU may also be used to drive one or more loudspeakers 420 of the smart phone.

Alternatively, the speaker may be connected to the CPU via the I/O bridge and the bus. Other components of the smart phone may be similarly connected via the bus, including a touch surface 432 such as a capacitive touch surface overlaid on the screen for the purposes of providing a touch input to the device, a microphone 434 for receiving speech from the user, one or more cameras 436 for capturing images, a global positioning system (GPS) unit 438 for obtaining an estimate of the smart phones geographical position, and wireless communication means 440.

The wireless communication means 440 may in turn comprise several separate wireless communication systems adhering to different standards and/or protocols, such as Bluetooth® (standard or low-energy variants), near field communication and Wi-Fi® as described previously, and also phone based communication such as 2G, 3G and/or 4G.

The systems are typically powered by a battery (not shown) that may be chargeable via a power input (not shown) that in turn may be part of a data link such as USB (not shown).

It will be appreciated that different smartphones may include different features (for example a compass or a buzzer) and may omit some of those listed above (for example a touch surface).

Thus more generally, in embodiments of the present invention a suitable mobile communication device such as smart phone 400 will comprise a CPU and a memory for storing and running an app, and wireless communication means operable to instigate and typically maintain wireless communication with the e-cigarette 10. It will be appreciated however that the mobile communication device may be any device that has these capabilities, such as a tablet, laptop, smart TV or the like.

Such a mobile communication device may also act as a bridge between the delivery device 10 and a remote device such as a server, by accessing the server over the internet via WiFi® or mobile data. Alternatively or in addition, the delivery device 10 may be capable of internet access on its own.

In embodiments of the description, an electronic vapor provision system ‘EVPS’ (e.g. a delivery device 10 or aerosol delivery device as described elsewhere herein) comprises the following.

Firstly, an electronic vapor provision device; this may be the delivery device itself or the vapor provision part thereof (e.g. cartomizer 30), as described elsewhere herein.

Secondly, at least a first sensor (22) configured to generate information about how firmly a user is holding the device. As described elsewhere herein, the sensor(s) may be placed at any suitable position for measuring how firmly a user is holding the device, and this position will typically relate to the morphology of the particular delivery device and how it is held in use. Similarly the orientation, shape, and/or size of the sensor may be chosen responsive to the morphology of the delivery device and how it is held in use. The sensor 22 is shown in FIG. 1 on the body of the delivery device, but it will be appreciated that this is a non-limiting example of the location, shape, and/or size of the sensor.

Thirdly, a control processor (e.g. processor 50 or CPU 410) configured to modify one or more operational parameters of the EVPS responsive to the information about how firmly the user is holding the device.

In some embodiments of the description, the at least first sensor 22 is not included with a user interface element of the electronic vapor provision system. Hence for example the sensor 22 is not part of an activation button or other UI element that provides intentional input to the EVPS from the user.

Rather, the sensor(s) are provided to determine how firmly a user is holding the device (e.g. grip strength on the device), typically within an anticipated part for the hand. Non limiting examples include a pincer or pen grip between thumb and fingers, where the sensor(s) may be located where the thumb and/or one or more fingers are anticipated to be located during such a grip; a fist grip between palm/finger base and fingers, where the sensor(s) may be located where some or all of the palm/finger base rests against the device and/or one or more fingers are anticipated to be located during such a grip; and/or an annular grip e.g. within curled forefinger and base of thumb, where the sensor(s) may be located where some or all of this annular grip is expected to occur e.g. during normal use.

For a pincer or pen-like grip, optionally the at least a first sensor is positioned to detect how firmly a user is holding the device in a pincer like grip on opposing complementary parts of the electronic vapor provision device.

For a fist-like grip, optionally the at least a first sensor is positioned to detect how firmly a user is holding the device in a fist like grip along at least part of a first axis of the electronic vapor provision device.

In some embodiments of the description, the sensor(s) may comprise a pressure sensor configured to measure a pressure applied to at least part of the electronic vapor provision device by the user's hold. Such a sensor directly detects the firmness with which the device (through the sensor) is being held. The advantage of a direct measurement is offset however by the need for the user to grip the device at least at the location of the sensor

Alternatively or in addition, the sensor(s) may comprise a strain sensor configured to measure a strain on at least part of the electronic vapor provision device created by the user's hold. Such a sensor indirectly detects the firmness with which the device is being held based on the elastic deformation of the device under the pressure of the hold. Whilst this measurement is indirect and requires previously determining (e.g. empirically) a relationship between holding press/grip strength and measured strain, it has the advantage that the deformation can be measured in the device without reliance on the user holding the device so as to apply pressure directly on the sensor.

Alternatively or in addition, the sensor(s) may comprise a capacitance sensor configured to measure a change in capacitance for at least part of the electronic vapor provision device created by the user's hold. Such a sensor indirectly measures how firmly the device is being held based on the change in capacitance of the sensor, which in turn corresponds to the surface area of the sensor that is in contact with the user's hand. Hence for example in a pen hold, if the device is being gripped firmly then the finger-tip area in contact with the sensor will increase and change the capacitance accordingly. Hence in effect a capacitance sensor detects the elastic deformation of the users hand under pressure (in contrast to a strain sensor detecting elastic deformation of the delivery device). A similar approach can be adopted based on a resistance sensor.

In embodiments of the present description, the at least first sensor is operably coupled to a flexible housing of at least part of the electronic vapor provision device. This enables the housing to elastically deformed in response to pressure from the users hold on the device, and for this to be detected for example by a pressure sensor directly beneath the housing, or a strain sensor mounted on the outer or inner surface of the housing.

Alternatively or in addition the at least first sensor is operably coupled to a flexible sleeve surrounding at least part of the electronic vapor provision device.

This enables the user to squeeze a device without requiring that the device itself is easily deformable (since the device typically comprises both the battery and a liquid reservoir, it is desirable that the device does not deform significantly in normal use, although it may nevertheless still elastically deform enough to be measured using a strain gauge as described elsewhere herein). Any of the sensor types described elsewhere herein may be incorporated into the flexible sleeve, or located beneath it, or in a window/opening in the sleeve.

Alternatively or in addition to determining how firmly the device is being gripped by the user's hands, in embodiments of the present description at least a first sensor is positioned to detect how firmly a user is holding the device mouthpiece between lips or teeth. Hence for example a pressure sensor, strain gauge, capacitance, and/or resistance sensor may be positioned at or near the mouthpiece to directly or indirectly detect the users grip on the device with their lips or teeth.

Such hand or mouth grip measurements can be used to determine whether or not the firmness of grip risks damaging the device and providing some form of feedback back to the user, for example in the form of a warning light or sound, or relaying a signal to a companion mobile device that provides such a warning message.

Alternatively or in addition, such hand or mouth grip measurements can be used to estimate a mood of the user. Hence in embodiments of the present description the control processor (50, 410) is configured to estimate a mood of the user based in part on how firmly the user is holding the device (in hand and/or mouth), and to modify one or more operational parameters of the EVPS based at least in part on the estimated mood.

Hence for example if an excessive grip is considered to indicate stress, then a larger amount of active ingredient per unit volume of inhaled air/vapor may be generated.

More generally, the modified one or more operational parameters of the EVPS may relate to one or more selected from the list consisting of:
i. a heating level of an aerosol generator of the EVPS;
ii. a heating profile of an aerosol generator of the EVPS;
iii. an amount of aerosol generated per unit of inhaled air;
iv. an amount of an active payload delivered per unit of inhaled air;
v. a selection of an active payload;
vi. an amount of flavoring delivered per unit of inhaled air;
vii. a selection of a flavoring;
viii. a combination of flavorings; and
ix. one or more user interface settings.

The choice of operational parameter and/or the change to that parameter may optionally depend on where the device is gripped (potentially as well as how hard it is gripped). Hence a fist like grip may be indicative of more stress than a pencil/pen like grip independent of how hard the grip is (and/or recognizing that a fist like grip may be inherently stronger than a pen grip and so the strength of grip for the two different holding positions is evaluated on different respective scales).

In this case, as a non-limiting example a stronger pen grip may change the amount of flavoring delivered, whilst a stronger for strip may change the amount of active payload.

Alternatively or in addition different operational parameters may be changed at different threshold strengths of grip; as a non-limiting example, above a first threshold the amount of flavoring may be increased, giving an illusion that the strength of the vapor has been increased and thus providing a placebo effect; however if the strength of grip exceeds a second, higher threshold amount, then the amount of active payload may be increased (optionally together with an increase in flavoring or further increase of flavoring), to actually provide increased strength for the vapor. It will be appreciated that different strategies may be chosen.

Hence more generally, in summary one or more sensors are configured to detect how firmly the user is holding their delivery device in either their hands or mouth. The or each sensor may be positioned according to an anticipated form of grip of the device, which may depend upon the morphology of the specific device. The or each sensor my detect grip strength directly or indirectly depending on the type of sensor and its location. A control processor uses the information for the or each sensor to determine how firmly a user is holding the device and is configured to modify one or more operational parameters in response. The choice of parameter and/or the modification thereof may depend upon the degree of firmness of hold (for example with respect to one or more thresholds) and/or the configuration of hold (for example pen versus fist holds). Thresholds of firmness may differ between configurations of hold.

As noted elsewhere herein, in embodiments of the description the role of the control processor may be implemented by one or more selected from the list consisting of a processor of the electronic vapor provision device, and a processor of a mobile communication device operably coupled to the electronic vapor provision device.

It will be appreciated that any suitable delivery device, aerosol provision system, or equally electronic vapor provision system, either alone or in conjunction with one or more partner devices such as a mobile communication device and/or a server, may implement the methods inherent in the operations of the systems described herein.

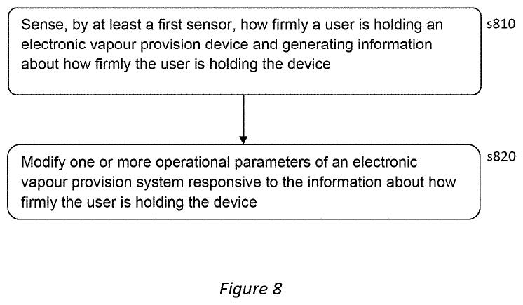

Accordingly, and turning now to FIG. 8, in a summary embodiment a non-therapeutic method of electronic vapor provision (e.g. a method of modifying one or more operational parameters of an EVPS) comprises the following operations.

Firstly, a sensing operation s810 of sensing by at least a first sensor how firmly a user is holding an electronic vapor provision device and generating information about how firmly the user is holding the device (e.g. based on the sensor output), as described elsewhere herein.

Secondly, a modifying operation s820 of modifying one or more operational parameters of an electronic vapor provision system ‘EVPS’ responsive to the information about how firmly the user is holding the device, as described elsewhere herein.

It will be apparent to a person skilled in the art that variations in the above method corresponding to operation of the various embodiments of the apparatus as described and claimed herein are considered within the scope of the present invention, including but not limited to that:
in an instance of the summary embodiment, the at least first sensor is not included with a user interface element of the electronic vapor provision system, as described elsewhere herein;
in an instance of the summary embodiment, the at least first sensor comprises a pressure sensor configured to measure a pressure applied to at least part of the electronic vapor provision device by the user's hold, as described elsewhere herein;
in an instance of the summary embodiment, the at least first sensor comprises a capacitance sensor configured to measure a change in capacitance for at least part of the electronic vapor provision device created by the user's hold, as described elsewhere herein;
in an instance of the summary embodiment, the at least first sensor comprises a resistance sensor configured to measure a change in resistance for at least part of the electronic vapor provision device created by the user's hold, as described elsewhere herein;
in an instance of the summary embodiment, the at least first sensor is operably coupled to a flexible sleeve surrounding at least part of the electronic vapor provision device, as described elsewhere herein;
in an instance of the summary embodiment, the at least a first sensor is positioned to detect how firmly a user is holding the device in a fist like grip along at least part of a first axis of the electronic vapor provision device, as described elsewhere herein;
in an instance of the summary embodiment, the at least a first sensor is positioned to detect how firmly a user is holding the device in a pincer like grip on opposing complementary parts of the electronic vapor provision device, as described elsewhere herein;
in an instance of the summary embodiment, the at least a first sensor is positioned to detect how firmly a user is holding the device mouthpiece between lips or teeth, as described elsewhere herein;
in an instance of the summary embodiment, the method comprises the operations of estimating a mood of the user based in part on how firmly the user is holding the device; and modifying one or more operational parameters of the EVPS based at least in part on the estimated mood, as described elsewhere herein;
in an instance of the summary embodiment, the modified one or more operational parameters of the EVPS relates to one or more selected from the list consisting of a heating level of an aerosol generator of the EVPS, a heating profile of an aerosol generator of the EVPS, an amount of aerosol generated per unit of inhaled air, an amount of an active payload delivered per unit of inhaled air, a selection of an active payload, an amount of flavoring delivered per unit of inhaled air, a selection of a flavoring, a combination of flavorings, and one or more user interface settings, as described elsewhere herein; and
in an instance of the summary embodiment, the modification of one or more operational parameters depends on where the device is gripped, as described elsewhere herein.

It will be appreciated that the above methods may be carried out on conventional hardware (such as EVPS 10, optionally in conjunction with mobile communication device 400 and/or server 1000) suitably adapted as applicable by software instruction or by the inclusion or substitution of dedicated hardware.

Thus the required adaptation to existing parts of a conventional equivalent device may be implemented in the form of a computer program product comprising processor implementable instructions stored on a non-transitory machine-readable medium such as a floppy disk, optical disk, hard disk, solid state disk, PROM, RAM, flash memory or any combination of these or other storage media, or realized in hardware as an ASIC (application specific integrated circuit) or an FPGA (field programmable gate array) or other configurable circuit suitable to use in adapting the conventional equivalent device. Separately, such a computer program may be transmitted via data signals on a network such as an Ethernet, a wireless network, the Internet, or any combination of these or other networks.

The foregoing discussion discloses and describes merely exemplary embodiments of the present invention. As will be understood by those skilled in the art, the present invention may be embodied in other specific forms without departing from the spirit or essential characteristics thereof. Accordingly, the disclosure of the present invention is intended to be illustrative, but not limiting of the scope of the invention, as well as other claims. The disclosure, including any readily discernible variants of the teachings herein, defines, in part, the scope of the foregoing claim terminology such that no inventive subject matter is dedicated to the public.

1-30. (canceled)

31. An electronic vapor provision system (EVPS), comprising:
an electronic vapor provision device;
at least a first sensor configured to generate information about how firmly a user is holding the device; and
a control processor configured to modify one or more operational parameters of the EVPS responsive to the information about how firmly the user is holding the device.

32. The EVPS of claim 31, wherein the first sensor is not included with a user interface element of the electronic vapor provision system.

33. The EVPS of claim 31, wherein the first sensor comprises a pressure sensor configured to measure a pressure applied to at least part of the electronic vapor provision device by the user's hold.

34. The EVPS of claim 31, wherein the first sensor comprises a strain sensor configured to measure a strain on at least part of the electronic vapor provision device created by the user's hold.

35. The EVPS of claim 31, wherein the first sensor comprises a capacitance sensor configured to measure a change in capacitance for at least part of the electronic vapor provision device created by the user's hold.

36. The EVPS of claim 31, wherein the first sensor comprises a resistance sensor configured to measure a change in resistance for at least part of the electronic vapor provision device created by the user's hold.

37. The EVPS of claim 31, wherein the first sensor is operably coupled to a flexible housing of at least part of the electronic vapor provision device.

38. The EVPS of claim 31, wherein the first sensor is operably coupled to a flexible sleeve surrounding at least part of the electronic vapor provision device.

39. The EVPS of claim 31, wherein the first sensor is positioned to detect how firmly a user is holding the device in a fist like grip along at least part of a first axis of the electronic vapor provision device.

40. The EVPS of claim 31, wherein the first sensor is positioned to detect how firmly a user is holding the electronic vapor provision device in a pincer like grip on opposing complementary parts of the electronic vapor provision device.

41. The EVPS of claim 31, wherein the first sensor is positioned to detect how firmly a user is holding the device mouthpiece between the lips or teeth of the user.

42. The EVPS of claim 31, wherein the control processor is configured to estimate a mood of the user based in part on how firmly the user is holding the device, and to modify one or more operational parameters of the EVPS based at least in part on the estimated mood.

43. The EVPS of claim 42, wherein the one or more operational parameters of the EVPS modified is selected from the group consisting of:
a heating level of an aerosol generator of the EVPS;
a heating profile of an aerosol generator of the EVPS;
an amount of aerosol generated per unit of inhaled air;
an amount of an active payload delivered per unit of inhaled air;
a selection of an active payload;
an amount of flavouring delivered per unit of inhaled air;
a selection of a flavouring;
a combination of flavourings; and
one or more user interface settings.

44. The EVPS of claim 42, wherein the one or more operational parameters modified depends on where the device is gripped.

45. The EVPS of claim 31, wherein the role of the control processor is implemented by one or more of:
a processor of the electronic vapor provision device; or
a processor of a mobile communication device operably coupled to the electronic vapor provision device.

46. A non-therapeutic electronic vapor provision method, comprising:
a sensing step by at least a first sensor wherein how firmly a user is holding an electronic vapor provision device is sensed and information generated about how firmly the user is holding the device; and
a modifying step wherein one or more operational parameters of an electronic vapor provision system ‘EVPS’ is modified responsive to the information about how firmly the user is holding the device.

47. The method of claim 46, wherein the first sensor is not included with a user interface element of the electronic vapor provision system.

48. The method of claim 46, wherein the first sensor comprises a pressure sensor configured to measure a pressure applied to at least part of the electronic vapor provision device by the user's hold.

49. The method of claim 46, wherein the first sensor comprises a strain sensor configured to measure a strain on at least part of the electronic vapor provision device created by the user's hold.

50. The method of claim 46, wherein the first sensor comprises a capacitance sensor configured to measure a change in capacitance for at least part of the electronic vapor provision device created by the user's hold.

51. The method of claim 46, wherein the first sensor comprises a resistance sensor configured to measure a change in resistance for at least part of the electronic vapor provision device created by the user's hold.

52. The method of claim 46, wherein the first sensor is operably coupled to a flexible housing of at least part of the electronic vapor provision device.

53. The method of claim 46, wherein the first sensor is operably coupled to a flexible sleeve surrounding at least part of the electronic vapor provision device.

54. The method of claim 46, wherein the first sensor is positioned to detect how firmly a user is holding the device in a fist like grip along at least part of a first axis of the electronic vapor provision device.

55. The method of claim 46, wherein the first sensor is positioned to detect how firmly a user is holding the device in a pincer like grip on opposing complementary parts of the electronic vapor provision device.

56. The method of claim 46, wherein the first sensor is positioned to detect how firmly a user is holding the device mouthpiece between lips or teeth of the user.

57. The method of claim 46, comprising the steps of:
estimating a mood of the user based in part on how firmly the user is holding the device; and
modifying one or more operational parameters of the EVPS based at least in part on the estimated mood.

58. The method of claim 57, wherein the one or more operational parameters of the EVPS modified is selected from the group consisting of:
a heating level of an aerosol generator of the EVPS;
a heating profile of an aerosol generator of the EVPS;
an amount of aerosol generated per unit of inhaled air;
an amount of an active payload delivered per unit of inhaled air;
a selection of an active payload;
an amount of flavouring delivered per unit of inhaled air;
a selection of a flavouring;
a combination of flavourings; and
one or more user interface settings.

59. The method of claim 46, wherein the one or more operational parameters modified depends on where the device is gripped.

60. A computer program comprising computer executable instructions adapted to cause a computer system to perform a method comprising:
a sensing step by at least a first sensor wherein how firmly a user is holding an electronic vapor provision device is sensed and information generated about how firmly the user is holding the device; and
a modifying step wherein one or more operational parameters of an electronic vapor provision system ‘EVPS’ is modified responsive to the information about how firmly the user is holding the device.
